(19) United States
(12) Patent Application Publication
(10) Pub. No.: US 2008/0312156 A1
(43) Pub. Date: Dec. 18, 2008

(54) In-Situ Crosslinkable Elastin-Like Polypeptides for Defect Filling in Cartilaginous Tissue Repair

(75) Inventors: Lori A. Setton, Durham, NC (US); Ashutosh Chilkoti, Durham, NC (US); Betre Helawe, Austin, TX (US); Kimberly T. Carlson, Durham, NC (US); Melissa McHale, Houston, TX (US); Dong Woo Lim, Ann Arbor, MI (US); Dana Nettles, Durham, NC (US)

Correspondence Address: MYERS BIGEL SIBLEY & SAJOVEC, PO BOX 37428, RALEIGH, NC, 27627, US

(73) Assignee: DUKE UNIVERSITY, DURHAM, NC (US)

(21) Appl. No.: 11/795,342

(22) PCT Filed: Jan. 17, 2006
(86) PCT No.: PCT/US06/01553

§ 371 Date: Aug. 22, 2008

Related U.S. Application Data
(60) Provisional application No. 60/644,867, filed on Jan. 18, 2005.
(60) Provisional application No. 60/680,180, filed on May 12, 2005.

(51) Int. Cl. A61K 38/00; A61P 19/00

(52) U.S. Cl. 514/15; 514/17; 514/18

(57) ABSTRACT

Defects in a cartilaginous tissue are filled by: (a) mixing (i) a first reagent composition preferably comprising an amine-free hydroxyalkyl (preferably hydroxymethyl) phosphine crosslinking agent with (ii) a second reagent composition comprising a bioelastic polymer, the bioelastic polymer preferably comprising elastomeric units, the elastomeric units preferably selected from the group consisting of bioelastic pentapeptides, tetrapeptides, and nonapeptides; to produce a therapeutic composition; and then (b) administering the therapeutic composition to the cargilagenous tissue. Compositions and kits for carrying out the method are also described.

### FIELD OF THE INVENTION

The present invention concerns methods, compositions and kits for repairing or filling defects in cartilaginous tissues.

### BACKGROUND OF THE INVENTION

Cartilaginous tissues play important roles in contributing to load support and energy dissipation in joints of the musculoskeletal system. These tissues include articular cartilage, the meniscus, and the intervertebral disc, which share the traits that they are predominantly avascular and alymphatic with a very low cell density. As a result, they exhibit a limited capacity for self-repair following injury or aging.

Tissue defects and/or degenerative changes associated with disorders such as arthritis, cartilage or meniscus trauma, aging-related disc disease, and congenital musculoskeletal disorders, may include fissures, tears, dessication (loss of hydration), biochemical changes, loss of cellularity, fibrillation, and calcification. Many of these tissue-level changes are believed to contribute to disability and symptoms that impair normal joint functions. Pathology related to these disorders cost our society an estimated $254 billion annually (Dorothy Rice, ScD (Hon), “Musculoskeletal Conditions: Impact and Importance,” presentation to Bone and Joint Decade Luncheon, Jun. 7, 2000). In addition, if left untreated, tissue defects and pathological changes may progress to end-stage joint disease, with few treatment options other than chronic use of anti-inflammatory agents, joint fusion, joint replacement, osteotomy or allograft transplantation.

As there are currently no satisfactory treatment options for these types of tissue failures, there is enormous interest in alternative strategies to replace or regenerate tissues, or promote repair towards the goal of inhibiting progressive joint disease. One class of strategies for tissue repair is based on the use of in situ forming biomaterials. These are low viscosity, fluid-like solutions that permit mixing with cells and/or bioactive factors, and may be polymerized or crosslinked in situ, usually via chemical or enzymatic initiation or the use of light. Thus, these polymers may readily fill and flow into an irregularly shaped defect, and be induced to undergo a fluid-solid transition in a controlled manner. Examples within this class of biomaterial that have been proposed for tissue defect filling include chitosan, poly(ethylene-glycol)-diacrylate, poly(vinyl alcohol), poly(N-isopropylacrylamide), and hyaluronic acid—methacrylate. To date, no biocompatible and non-immunogenic biomaterials have been engineered with physical properties appropriate to promote cartilaginous tissue defect regeneration or repair.

U.S. Pat. No. 6,699,294 to Urry describes polymers of elastin-like peptides. In situ crosslinking is briefly discussed but reagents useful for carrying out effective in situ cross-linking are not described, and particularly does not suggest how to form relatively stiff materials in situ. (see also U.S. Pat. No. 4,589,882 to Urry; U.S. Pat. No. 6,753,311 to Fertala and Ko; and U.S. Pat. No. 6,063,061 to Wallace).

M. Knight et al., Evaluation of Enzymatically Crosslinked Elastin-like Polypeptide Gels for Cartilage Tissue Repair (2003 Summer Bioengineering Conference, June 25-29, Sonesta Beach Resort in Key Biscayne, Fla.) described ELP polymers cross-linked with an enzymatic crosslinking agent, human transglutaminase. (See also M. Knight et al., Enzymatically Crosslinked Elastin-like Polypeptide Gels for Cartilage Tissue Repair (abstract) (Tissue Engineering Society International 6^{th} Annual International Conference & Exposition, Dec. 10-13, 2003, Orlando, Fla.). However, the biomechanical properties of polymers crosslinked with human tissue transglutaminase were not particularly advantageous.

K Trabbic-Carlson et al., Biomacromolecules 4, 572-580 (2003) describes the swelling and mechanical behaviors of chemically cross-linked hydrogels of elastin-like polypeptides with tris-succinimidyl aminotriacetate (TSAT) as a cross-linking agent, but this cross-linking agent is not suitable for use in situ (see also H. Betre et al., Characterization of a Genetically Engineered Elastin-like Polypeptide for Cartilaginous Tissue Repair Biomacromolecules 3, 910-916 (2002)).

Hence, there remains a need for alternative strategies to either replace tissues or promote repair.

### SUMMARY OF THE INVENTION

A first aspect of the present invention is a method of filling a defect in a cartilaginous tissue, comprising the steps of: (a) mixing (i) a first reagent composition preferably comprising an amine-free hydroxyalkyl (preferably hydroxymethyl) phosphine crosslinking agent with (ii) a second reagent composition comprising a bioelastic polymer, the bioelastic polymer preferably comprising elastomeric units, the elastomeric units preferably selected from the group consisting of bioelastic pentapeptides, tetrapeptides, and nonapeptides; to produce a therapeutic composition; and then (b) administering the therapeutic composition to the cargilagenous tissue (typically, in an amount sufficient to at least partially fill the defect with the therapeutic composition, with the elastin-like polypeptides crosslinking within the defect).

A second aspect of the invention is a kit useful for making a composition of filling defects in cartilageneous tissue, comprising: (i) a first reagent composition preferably comprising an amine-free hydroxyalkyl (preferably hydroxymethyl) phosphine crosslinking agent; and (ii) a second reagent composition comprising a bioelastic polymer, the bioelastic polymer preferably comprising elastomeric units, the elastomeric units preferably selected from the group consisting of bioelastic pentapeptides, tetrapeptides, and nonapeptides.

A third aspect of the invention is a therapeutic composition produced by the process of mixing (i) a first reagent composition preferably comprising an amine-free hydroxyalkyl (preferably hydroxymethyl) phosphine crosslinking agent with (ii) a second reagent composition comprising a bioelastic polymer, the bioelastic polymer preferably comprising elastomeric units, the elastomeric units preferably selected from the group consisting of bioelastic pentapeptides, tetrapeptides, and nonapeptides, to produce the therapeutic composition. The composition is preferably sterile and is preferably provided in a suitable administration device such as a syringe for injection.

In general, bioelastic polymers produced by the methods, kits and compositions described herein have, when crosslinked, mechanical properties suitable for their intended use, and are generally characterized by: (i) a complex modulus |G*| of 0.1 or 1 to 650 or 700 kPa; (ii) a loss angle δ of 1 or 2 to 50°; and (iii) an equilibrium shear modulus μ of 0.1 to 500 kPa.

In some embodiments the cross-linking agent has a free carboxylic acid group (which is available for coupling a compound of interest thereto). In some embodiments the cross-linking agent has a compound of interest coupled thereto (e.g., via the previously free carboxylic acid group).

A further aspect of the present invention is the use of a bioelastic polymer as described herein for the preparation of a composition or medicament for carrying out a method as described herein.

A still further aspect of the present invention is the use of a crosslinking agent as described herein for the preparation of a composition or medicament for carrying out a method as described herein.

The foregoing and other objects and aspects of the present invention are explained in greater detail in the specification set forth below.

### BRIEF DESCRIPTION OF THE DRAWINGS

FIG. 1. Schematic presentation of inter-, or intra-molecular crosslinking mechanism between Lys residues of ELPs and THPP (β-[tris(hydroxymethyl) phosphino]-propionic acid) through Mannich type condensation reaction. Primary amine groups of Lys side chains at predetermined interval of ELPs can be crosslinked in the wide range of pH.

FIG. 2. Average percentage of viable cells counted for each formulation after 3 days, 2 or 4 weeks of culture.

FIG. 3. Mechanical property data presented as the mean +/− standard deviation.

FIG. 4A-4B. Oscillatory Theological profiles when 20 (wt-%) of THPP crosslinker in phosphate buffer (pH7.5) at 700 mM of NaCl was added to 21 (wt-%) of ELP [KV7F-144] in phosphate buffer (pH 7.5) prepared onto a bottom platen of which temperature was equilibrated at 35° C. Elastic modulus, G′ and viscous modulus, G″ during crosslinking are shown in A and B, respectively. The rheological behaviors during crosslinking were characterized in dynamic, torsional shearing using a cone-on-plate configuration (ARES Rheometer, TA Instruments, cone angle=0.1 rad, plate radius=25 mm) at 35° C. (1 Hz frequency and 0.01 maximum shear strain). The crosslinking of ELP [KV7F-144] at 35° C. came to completion within one minute through the condensation of Lys residues and THPP as indicated by the crossover of the elastic modulus, G′ and the viscous modulus, G″ and also the elastic modulus, G′ increased over 5 kPa in one hour at 35° C. as indicated in B.

FIG. 5A-5C. Mechanical properties of ELP[KV7F-72] and ELP[KV2F-64] gels crosslinked at pH 7.5 and 12.0. Equilibrium compressive modulus, E_{eq} (kPa), complex shear modulus, G* (kPa), and equilibrium shear modulus, μ (kPa) were shown in A, B and C, respectively. Mechanical tests from the fully crosslinked ELP gels were obtained at 23° C. on a strain-controlled rheometer (ARES; Rheometrics Scientific, Piscataway, N.J.) in a parallel plate configuration (plate radius=10 mm; stainless steel porous platens; 50% porosity; 40-60 mm pore size). E_{eq} was measured after consecutive compressive stress relaxation tests of nominal strains of 5, 10 and 15% and at 15% compression, G* was obtained from a frequency sweep test (0.1 to 50 rad/sec frequency; 0.01 maximum shear strain). Equilibrium shear modulus, μ was calculated from linear regression of normal stress (σ) at equilibrium on strain (ε) at 5% strain increments from 5-15%.

FIG. 6. Photographic images of ELP[KV7F-72] hydrogels crosslinked at pH 7.5 (right) and pH 12.0 (left). Bar, 5 mm.

FIG. 7. Live/Dead assay of fibroblasts in crosslinked (A) single- and (B) tri-block ELP hydrogels at day 3. Fibroblasts were stained green in the cell viability assay, suggesting the crosslinking reaction was not cytotoxic to fibroblasts at 3 day, but cell viability based on DNA quantitation was found to be less than 50% immediately after crosslinking, but was greater than 60% after 3 days of culture, values that compare well with embedding cells in alginate or agarose.

FIG. 8: Representative MR image of left (unfilled) and right knee (ELP filled) of goat 6 months after surgery showing appearance of osteochondral defects filled and unfilled with ELP solution. Defects are evident within bony areas and at articular surface. Images were acquired at ˜30 degrees of flexion on a 7.1 T scanner (Magnex Scientific) using a 5 cm-diameter birdcage RF coil (Center for In Vivo Microscopy, Duke University). Images shown are representative 2D slices acquired with a GRE sequence (6 cm FOV, 256×256 matrix size, 500 ms TR, 12 ms TE, 60 degree flip, 8 averages, interleaved 1.5 mm slice thickness acquisition).

FIG. 9: Summary of MRI grading of joints 3 months (top) and 6 months (bottom) after surgery. Note that MRI imaging grades represent average values for n=2 (3 mos) and n=4 (6 mos) animals. * significantly different from ELP-filled.

FIG. 10: Summary of histological grading of cartilage from goat knees at 3 months (top) and 6 months (bottom) after surgery. * significantly different from ELP-filled.

FIG. 11 shows representative stained sections of cartilage from one goat at 6 months after surgery. Defect sites are evident where underlying bone is more disorganized and/or of increased density.

### DETAILED DESCRIPTION OF THE PREFERRED EMBODIMENTS

“Alkyl” as used herein refers to C1-C6 alkyl, such as methyl, ethyl, propyl, isopropyl, butyl, tert-butyl, sec-butyl, pentyl, hexyl, etc.

“Articular cartilage” as used herein as used herein refers to cartilage associated with a joint, particularly the knee, hip, or elbow.

“Meniscus” as used herein refers to both the medial meniscus of the knee joint and the lateral meniscus of the knee joint.

“Intervertebral disc” as used herein refers to a layer of cartilaginous material between vertebrae of the spinal column, and includes both the nucleus pulposus and the anulus fibrosus.

“Nucleus pulposus” as used herein refers to a semifluid mass of fine what and elastic fibers that forms the central portion of the intervertebral disc.

“Bioelastic polymer” as used herein refers to compounds that comprise repeating elastomeric units. The elastomeric units are typically pentapeptides, tetrapeptides, or nonapeptides.

“Defect” as used herein with respect to cartilaginous tissue is meant to encompass any disruption in the structural continuity or integrity of that tissue within its intended anatomic environment. Hence defects include both surface defects and internal defects. Examples of defects include but are not limited to incisions, rips, tears and/or lacerations; openings, holes, herniations, and/or lacunae; worn and/or abraded surfaces, compressed structures (particularly disks), etc.

“Phosphine” as used herein refers to PH₃ and compounds derived from it by substituting, one, two or three hydrogen atoms by hydrocarbyl groups R₃P.

“Subjects” as used herein includes human subjects (including both male and female subjects and including juvenile, adolescent, adult, and geriatric subjects), as well as animal subjects, particularly other mammalian subjects such as primates, dogs, cats, and horses, for veterinary purposes.

The disclosures of all United States patent references cited herein are to be incorporated by reference herein in their entirety.

Cross-Linking Agents. Cross-linking agents used to carry out the present invention are preferably amine-free compounds containing hydroxymethyl phosphines (>P—CH₂—OH) that react with primary and secondary amines, generating stable aminomethylphosphines (>P—CH₂—N<) and only water as a byproduct of the crosslinking reaction via Mannich type condensation reaction. The crosslinking agents are amine-free compounds containing hydroxyalkyl, preferably hydroxymethyl, phosphine (HMP) groups, as described in U.S. Pat. No. 5,948,386 to Katti et al. In one embodiment, such compounds have the basic formula, hydroxymethyl phosphines (HMP): R—P(CH₂OH)₂, where R is any suitable organic molecule or group, such as —CH₂OH, —H, —C₂H₅, —C₆H₅, —COOH, —COOCH₃, —COOC₂H₅, etc. For example, tris(hydroxymethylphosphine), P(CH₂OH)₃ (or “THP”) is the basic crosslinker for this basic category. In a second embodiment, such compounds have the complex formula that contains at least one or more of the basic formula, hydroxymethylphosphines (HMP): R—P(CH₂OH)₂, where R is any suitable organic molecule or group, such as —CH₂OH, —H, —C₂H₅, —C₆H₅, —COOH, —COOCH₃, —COOC₂Z₅, etc. For example, this group includes combined molecules of the two basic hydroxymethyl phosphines, 1,2-bis(bis(hydroxymethyl)phosphino)-benzene (HMPB), 1,2-bis(bis(hydroxymethyl)phosphino) ethane (HMPE) that are converted from mono and multiprimary phosphines, 1,2-bis(phosphino)-benzene, 1,2-bis(phosphino)ethane (PE). In a third embodiment, such compounds include homo- and hetero-functional crosslinking agents where the molecules having the hydroxymethylphosphine group are attached to the same as the hydroxymethyl phosphine containing molecules and to the other chemically active groups different from the hydroxymethyl phosphine groups. For example, this compound includes beta-(tris-(hydroxymethyl)phosphino) propionic acid (THPP).

Thus particular examples of suitable cross-linking agents include tris(hydroxymethyl)phosphine (THP) and B-(tris-(hydroxymethyl)phosphino) propionic acid (THPP):

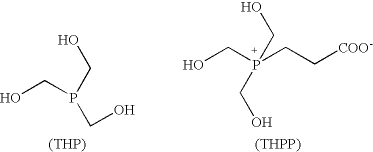

In a preferred embodiment the cross-linking agent includes a free carboxylic acid group (—COOH), which is useful for linking compounds of interest to the cross-linking agent before or after polymerization, to thereby link the compound of interest to the bioelastic polymer.

Bioelastic Polymers. Bioelastic polymers are known and described in, for example, U.S. Pat. No. 5,520,672 to Urry et al. In general, bioelastic polymers are polypeptides comprising elastomeric units of bioelastic pentapeptides, tetrapeptides, and/or nonapeptides. Thus in some embodiments the elastomeric unit is a pentapeptide, in other embodiments the elastomeric unit is a tetrapeptide, and in still other embodiments the elastomeric unit is a nonapeptide. Bioelastic polymers that may be used to carry out the present invention are set forth in U.S. Pat. No. 4,474,851, which describes a number of tetrapeptide and pentapeptide repeating units that can be used to form a bioelastic polymer. Specific bioelastic polymers that can be used to carry out the present invention are also described in U.S. Pat. Nos. 4,132,746; 4,187,852; 4,500,700; 4,589,882; and 4,870,055. Still other examples of bioelastic polymers are set forth in U.S. Pat. No. 6,699,294 to Urry, U.S. Pat. No. 6,753,311 to Fertala and Ko; and U.S. Pat. No. 6,063,061 to Wallace.

In one embodiment, the bioelastic polymers used to carry out the present invention are polypeptides of the general formula (VPGXG)ₘ where X is any amino acid other than proline (e.g., Ala, Leu, Phe) and m is any suitable number such as 2, 3 or 4 up to 60, 80 or 100 or more. The frequency of the various amino acids including K as the fourth amino acid can be changed, as well as the frequency of X. For example, the bioelastic polymers used to carry out the present invention may be polypeptides of the general formula: [(VPGXG)ₘ(VPGKG)ₙ]ₒ, where m is 2, 3 or 4 to 20 or 30, n is 1, 2 or 3, o is at least 2, 3 or 4 up to 30, 40 or 50 or more. Any ratios of X/K can be possible, which means where m is 1, 2, or 3 up to 100, 150, or 300 or more, n is 1, 2 or 3 up to 100 or 150 or 300 or more, o is at least 1, 2, or 3 up to 100, 150 or 300 or more.

For example, bioelastic polymers used to carry out the present invention may comprise repeating elastomeric units selected from the group consisting of bioelastic pentapeptides and tetrapeptides, where the repeating units comprise amino acid residues selected from the group consisting of hydrophobic amino acid and glycine residues and where the repeating units exist in a conformation having a beta-turn of the formula:

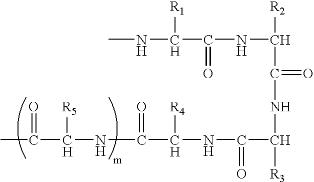

wherein R₁—R₅ represent side chains of amino acid residues 1-5, and m is 0 when the repeating unit is a tetrapeptide or 1 when the repeating unit is a pentapeptide. Nonapeptide repeating units generally consist of sequential tetra- and pentapeptides. Preferred hydrophobic amino acid residues are selected from the group consisting of alanine, valine, leucine, isoleucine, proline, phenylalanine, tryptophan, and methionine. In many cases, the first amino acid residue of the repeating unit is a residue of valine, leucine, isoleucine or phenylalanine; the second amino acid residue is a residue of proline; the third amino acid residue is a residue of glycine; and the fourth amino acid residue is glycine or a very hydrophobic residue such as tryptophan, phenylalanine or tyrosine. Particular examples include the tetrapeptide Val-Pro-Gly-Gly, the tetrapeptide GGVP, the tetrapeptide GGFP, the tetrapeptide GGAP, the pentapeptide is Val-Pro-Gly-Val-Gly, the pentapeptide GVGVP, the pentapeptide GKGVP, the pentapeptide GVGFP, the pentapeptide GFGFP, the pentapeptide GEGVP, the pentapeptide GFGVP, and the pentapeptide GVGIP. See, e.g., U.S. Pat. No. 6,699,294 to Urry.

Design Criteria. Physical properties of the repairing cartilaginous tissues are important for determining the clinical success of the repair (Scientific Recommendations, NIH BECON 2001 meeting on “Reparative Medicine: Growing Tissues and Organs”, Guilak et al. 2002). One approach to functional tissue repair is based on rapid restoration of the physical properties of the defect site in order to provide for mechanical weight-bearing, load transfer and nutrient diffusion to cells. Sets of physical properties for native articular cartilage, meniscus, and intervertebral disc have been obtained through mechanical testing in tension, compression, shear and combined loading configurations (Table 1), as well as direct tests of diffusion, permeability, and electrical conductivity. These property datasets serve as design guidelines for synthesizing an appropriate scaffold for cartilage defect filling in accordance with the present invention.

(i) Equilibrium Shear Modulus. Some of the most comprehensive datasets for cartilaginous tissue properties were obtained from testing in torsional or simple shear. Cartilaginous tissues resist shearing stresses and strains through stretching and deformation of the collagen-proteoglycan matrix. The equilibrium shear modulus, μ, provides a measure of the shear stress (τ) generated in response to a unit increment of shear strain (γ, where τ=μγ) measured at equilibrium (i.e., after all transient, or viscoelastic effects are eliminated). Data for μ has been determined by linear estimation over the range of torsional shear strains, γ=0.01-0.2. Typically, cylindrical samples of articular cartilage, meniscus, annulus fibrosus or other soft tissue are prepared and placed between two platens of a dynamic mechanical spectrometer (ARES, Rheometrics Scientific). Torsional shear strains may be applied to the sample and the resulting torque measured for calculation of the shear stress from sample geometry. The linear relationship between shear stress and strain is defined as the equilibrium shear modulus.

TABLE 1
Equilibrium and dynamic shear properties of cartilaginous
tissues. Ranges represent the 95% confidence intervals
based on available property data.

| TISSUE | \|G*\|(kPa) | δ (°) | μ (kPa) | Ref. |
|---|---|---|---|---|
| Nucleus | 0.1-70 | 2-50 | 0.1-30 | Iatridis et al. 1997, Bodine et |
| Pulposus | | | | al. 1982 |
| Anulus | 1-400 | 2-40 | 10-210 | Fujita et al. 2000, latridis et |
| Fibrosus | | | | al. 1999, Bodine et al. 1982 |
| Meniscus | 2-650 | 2-30 | 2-500 | Anderson et al. 1991; Zhu et |
| | | | | al. 1994; Mow et al. 1992 |
| Articular | 2-400 | 2-20 | 2-400 | Hayes and Mockros 1971; |
| Cartilage | | | | Zhu et al. 1986; Hayes & |
| | | | | Bodine 1978, Zhu et al. 1992, |
| | | | | Setton et al. 1995; Woo et al. |
| | | | | 1987 |

(ii) Complex Modulus and Loss Angle. The magnitude of the complex modulus, |G*|, provides a measure of the shear stress generated under dynamic shearing strain conditions (γ=γ₀e^{iωt}, and τ=τ₀e^{i(ωt+δ)}). The ratio of resulting stress to strain is given by τ₀/γ₀(cos δ+isin δ) with real and imaginary components, G′ and G″, respectively. Note that the magnitude of the complex modulus is defined as |G*|=sqrt (G′²+G″²), and the value δ represents the dissipation inherent in the material (loss angle, δ=0° for an elastic solid; δ=90° for a viscous fluid). The magnitude of the complex modulus and loss angles have been measured for cartilaginous tissues over a range of frequencies, ω, that spans 0.001-1000 rad/s, but in some cases as high as 800 Hz. Tests of controlled torsional shear strain or shear stress have been used as described above.

The compressive properties of cartilaginous tissues have also been obtained from testing in uniaxial compression or indentation (compressive modulus, H_{A}) or from unconfined compression (compressive modulus, E). Cartilaginous tissues resist compressive stresses and strains through a partitioning of the load between fluid (water) and the collagen-proteoglycan matrix. The equilibrium compressive moduli, E or H_{A}, provide measures of the compressive stress (a) generated in response to a unit increment of compressive strain (ε) measured at equilibrium (i.e., after all transient, or viscoelastic effects are eliminated). The transient stress-relaxation behaviors are partly described by the fluid-flow permeability coefficient (k). As for the shear testing, cylindrical specimens are often prepared arid tested between two platens of a confining chamber or radially unconstrained test chamber. Relationships between sample geometry and applied compressive stresses or strains and resulting force-displacement data are used to calculate the material properties. In the case of articular cartilage, indentation testing has also been used where the compressive load is applied via a loading platen (indenter) that contacts the surface over a limited, smaller region. Data for the moduli have been determined by linear estimation up to ε=0.25 (Table 2).

Diffusion Coefficients. Diffusion of solutes and metabolites through cartilaginous tissues is held to be important for sustaining the local cell population. The diffusion coefficients of cartilage (D) have been estimated from biochemical tracking of applied solute gradients over time, and from estimates of water or solute diffusion using fluorescence or magnetic resonance imaging. Solutes commonly used have included radiolabeled or fluorescently labeled tracer ions, uncharged solutes (e.g., sucrose) and larger molecular weight molecules including growth factors, BSA and dextrans. The diffusion coefficient provides an estimate of the distance traveled by a solute per second within the cartilage matrix (Table 3). Thus, diffusion coefficients larger than those given for native tissues would be expected to provide for maintained or even enhanced transport of nutrients and metabolites.

TABLE 2
Equilibrium compressive properties of cartilaginous tissues. Ranges represent
the 95% confidence intervals based on available property data.

| | | | | | k₀ | |
|---|---|---|---|---|---|---|
| TISSUE | E (kPa) | H_{A} (kPa) | \|E*\|(kPa) | δ (°) | (m⁴/N · s) × 10¹⁵ | Ref. |
| Nucleus | NA | NA | 30-120 | 10-40 | NA | Leahy and Hukins |
| Pulposus | | | | | | 2001 |
| Anulus | NA | 5-1000 | NA | NA | 0.01-0.75 | Best et al. 1994; |
| Fibrosus | | | | | | Iatridis et al. 1998; |
| | | | | | | Houben et al. 1997 |
| Meniscus | 5-100 | 5-500 | NA | NA | 0.6-10 | Joshi et al. 1995; |
| | | | | | | Proctor et al 1989; |
| | | | | | | Zhu et al. 1994; |
| | | | | | | Mow et al. 1992 |
| Articular | 5-2000 | 5-1000^{a} | | | 0.5-5.0^{a} | Chahine et al. 2004; |
| Cartilage | | | | | | Armstrong |
| | | | | | | and.Mow 1982, |
| | | | | | | Athanasiou et al. |
| | | | | | | 1991, Mow et al. |
| | | | | | | 1995, Setton et al. |
| | | | | | | 1995; Sokoloff |
| | | | | | | 1966; Kempson et |
| | | | | | | al. 1972; Parsons & |
| | | | | | | Black 1977; Hayes |
| | | | | | | and Mockros 1971 |
| | | | 100-50,000 | 1-50 | | Park et al. 2004 |

^{a}Property estimated but not experimentally verified.

0.1-10 Hz at a temperature of 37 degrees C. Freezing appeared to increase the storage modulus, E′, but not the loss modulus, E″, or tan delta=E″/E′. These parameters, E′, E″ and tan delta, had values of 64+/−28 kPa, 24+/−11 and 0.33 kPa+/−0.07, respectively. The value of tan delta passed through a minimum at a loading frequency of 0.9+/−0.2 Hz. The water content of the specimens was 80+/−2%.

TABLE 3
Diffusion coefficients.

| | Diffusion Coefficent | |
|---|---|---|
| TISSUE | of ~70 kDa solutes (μm²/s) | Reference |
| Intervertebral | 5-25 | Urban and Maroudas, 1980 |
| Disc | | |
| Meniscus | N/A | N/A |
| Articular | 25-45 | Leddy and Guilak, 2003 |
| Cartilage | | |
| (middle zone) | | |

Compositions and Formulations. A first reagent composition of the invention comprises a crosslinking agent in a suitable (preferably sterile) container such as a vial, syringe, jar, polymer package or the like. The crosslinking agent may be provided in hydrated form or nonaqueous form. A second reagent composition of the invention typically comprises a bioelastic polymer in a suitable (preferably sterile) container such as a vial, syringe, jar, polymer package or the like. the bioelastic polymers may likewise be provided in hydrated form, dehydrated form, or lyophilized form.

Either, or both, of the first and second reagent compositions of the present invention may be further combined with other materials and components, such as bioactive component(s) to be delivered to the patient, viscosity modifiers, such as carbohydrates and alcohols, and other materials intended for other purposes, such as to control the rate of resorption. Exemplary bioactive components include, but are not limited to, proteins, carbohydrates, nucleic acids, and inorganic and organic biologically active molecules such as enzymes, antibiotics, antineoplastic agents, bacteriostatic agents, bacteriocidal agents, antiviral agents, hemostatic agents, local anesthetics, anti-inflammatory agents, hormones, antiangiogenic agents, antibodies, neurotransmitters, psychoactive drugs, drugs affecting reproductive organs and oligonucleotides, such as antisense oligonucleotides. Such bioactive components will typically be present at relatively low concentrations, typically below 10% by weight of the compositions, usually below 5% by weight, and often below 1% by weight. Exemplary hemostatic agents include thrombin, fibrinogen and clotting factors. Hemostatic agents like thrombin may be added in concentrations ranging from 50 to 10,000 Units thrombin per ml gel, preferably from about 100 Units thrombin per ml gel to about 1000 Units thrombin per ml gel. See, e.g., U.S. Pat. No.6,063,061.

A kit of the present invention will generally comprise a first reagent composition and a second reagent composition, each in a separate sterile container as described above, with the two together typically combined in an overall container such as a box, jar, pouch, tray, or the like. The kit may include written instructions for carrying out methods as described herein, which written instructions may be printed on a separate sheet of paper or other material and packaged on or within the container or may be printed on the container itself. The bioelastic polymer reagent composition may be provided in hydrated, dehydrated or lyophilized form. The kit may optionally include a separate container with a suitable aqueous buffer for hydration. Other system components such as the applicator, e.g. syringe, may also be provided.

Compounds of Interest. Any suitable compound, including but not limited to proteins and peptides, may be coupled to the crosslinking agent via the carboxylic acid group. In general, the compounds of interest may be detectable groups (e.g., for monitoring the crosslinked material) or a therapeutic group.

For example, the compound of interest may be selected from the group of bone morphogenetic proteins, peptides, and growth factors, more particularly human calcitonin analogs, osteogenic growth peptides, and osteogenic growth peptide-human calcitonin analog hybrids. See, e.g., U.S. Pat. No. 6,593,394.

The compound of interest may be selected from the group of antiinfectives such as antibiotics and antiviral agents; chemotherapeutic agents (i.e. anticancer agents); anti-rejection agents; analgesics and analgesic combinations; anti-inflammatory, agents; hormones such as steroids; growth factors, including bone morphogenic proteins (i.e. BMP's 1-7), bone morphogenic-like proteins (i.e. GFD-5, GFD-7 and GFD-8), epidermal growth factor (EGF), fibroblast growth factor (i.e. FGF 1-9), platelet derived growth factor (PDGF), insulin like growth factor (IGF-I and IGF-II), transforming growth factors (i.e. TGF-.beta. I-III), vascular endothelial growth factor (VEGF); and other naturally derived or genetically engineered proteins, polysaccharides, glycoproteins, or lipoproteins. See, e.g., D. Overaker, U.S. Pat. No. 6,575,986.

The compound of interest may be compounds or agents that actually promote or expedite healing, the effectors may also include compounds or agents that prevent infection (e.g., antimicrobial agents and antibiotics), compounds or agents that reduce inflammation (e.g., anti-inflammatory agents), compounds that prevent or minimize adhesion formation, such as oxidized regenerated cellulose (e.g., INTERCEED and Surgicel.RTM., available from Ethicon, Inc.), hyaluronic acid, and compounds or agents that suppress the immune system (e.g., immunosuppressants). Suitable compounds of interest include heterologous or autologous growth factors, proteins (including matrix proteins), peptides, antibodies, enzymes, platelets, glycoproteins, hormones, cytokines, glycosaminoglycans, nucleic acids, analgesics, viruses, virus particles, and cell types. It is understood that one or more effectors of the same or different functionality may be incorporated within the scaffold. Suitable compounds of interest include the multitude of heterologous or autologous growth factors known to promote healing and/or regeneration of injured or damaged tissue. Suitable compounds of interest include chemotactic agents; therapeutic agents (e.g., antibiotics, steroidal and non-steroidal analgesics and anti-inflammatories, anti-rejection agents such as immunosuppressants and anti-cancer drugs); various proteins (e.g., short term peptides, bone morphogenic proteins, glycoprotein and lipoprotein); cell attachment mediators; biologically active ligands; integrin binding sequence; ligands; various growth and/or differentiation agents and fragments thereof (e.g., epidermal growth factor (EGF), hepatocyte growth factor (HGF), IGF-I, IGF-II, TGF-.beta. I-III, growth and differentiation factors, vascular endothelial growth factors (VEGF), fibroblast growth factors (FGF), platelet derived growth factors (PDGF), insulin derived growth factor (IGF) and transforming growth factors, parathyroid hormone, parathyroid hormone related peptide, bFGF; TGFB superfamily factors; BMP-2; BMP-4; BMP-6; BMP-12; sonic hedgehog; GDF5; GDF6; GDF8; MP52, CDMP1); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments and DNA plasmids. Suitable effectors likewise include the agonists and antagonists of the agents noted above. The growth factor can also include combinations of the growth factors listed above. In addition, the growth factor can be autologous growth factor that is supplied by platelets in the blood. In this case, the growth factor from platelets will be an undefined cocktail of various growth factors. See, e.g., J. Hwang et al., US Patent Application 20040267362

Methods of Use. In use, the two reagent compositions will be mixed together (e.g., by injecting one into a container containing the other, by repeatedly passing through a syringe, by mechanical aggitation, etc.) to prepare a composition (typically a hydrogel) for administration to a subject. The composition for administration will, in general, comprise or consist essentially of from 0.01 to 5 percent by weight of crosslinking agent, from 5 to 50 percent by weight bioelastic polymer, and from 50 to 95 percent by weight water (with other minor or ancillary ingredients permissible as noted above). The amount prepared for administration will depend upon the size and type of the defect being filled but will generally be from 1 to 100 or 200 milliliters.

The compositions of the invention may be administered by any suitable means, including but not limited to a syringe, a spatula, a brush, a spray, manually by pressure, etc. In a particular embodiment the compositions may be pre-mixed and applied by injection using a syringe or the like. For an intervertebral disk, administration or filling may be by injection through a syringe of, e.g., 16 to 20 gauge. This procedure may be performed via minimally invasive surgical approaches including arthroscopy and subcutaneous injection approaches.

In one embodiment, the filling step is carried out within one or two hours of the mixing step. In other embodiments the mixing step may be carried out an then an intervening freezing or freeze-drying step carried out prior (and/or a step of lowering the pH), after which the mix may be thawed and/or rehydrated and/or pH adjusted to raise the pH and the administration or filling step then carried out. The particular choice of mixing just prior to use or mixing and then storing will depend upon the particular end usage, circumstances of the procedure, etc., with one approach being more convenient in one situation and another approach being more convenient in others.

While the present invention has been explained primarily with reference to defect filing in cartilaginous tissue, it will be appreciated that the methods, kits, compositions and materials of the invention can also be used for any of the variety of purposes for which bioelastic materials have been described.

Proposed Mechanism. Without wishing to be bound to any specific theory of the invention, the following is provided for general guidance herein.

The reactions of hydroxymethylphosphines with amino acids, peptides, and proteins have been studied for biomedical and chemical applications, only in terms of an incorporation of phosphines onto diverse peptide backbones to coordinate with transition metals and radiometals for radiopharmaceuticals of cancer therapy and an enzyme immobilization by Mannich type condensation reaction of hydroxymethylphosphine groups with amines of proteins [Berning, D. E., et al., Chemical and biomedical motifs of the reactions of hydroxymethylphosphines with amines, amino acids, and model peptides. Journal Of The American Chemical Society, 1999. 121(8): p. 1658-1664; Petach, H. H. et al., J. Chem. Soc. Chem. Comm., 1994(18): p. 2181-2182; Berning, D. E., et al., Au-198-labeled hydroxymethyl phosphines as models for potential therapeutic pharmaceuticals. Nuclear Medicine And Biology, 1998. 25(6): p. 577-583 (1998)]. Katti and Volkert have studied that P—C bonds of hydroxymethylphosphines can be easily introduced into various structures of mono, multiprimary phosphines (R—PH₂, where R is any suitable organic groups, such as —CH₂OH, —H, —C₂H₅) by formylation of their P—H bonds, producing diverse structures with hydroxymethylphosphines (R—P(CH₂OH)₂) positioned at a predetermined site for its own applications [Katti, K. V. et al., New vistas in chemistry and applications of primary phosphines, in New Aspects In Phosphorus Chemistry Iii. 2003. p. 121-141]. It has been shown that in terms of its biological activity and function, gold complexes with ligands of water soluble and air stable hydroxymethylphosphines including tris(hydroxylmethyl)phosphine (THP), 1,2-bis[bishydroxymethylphosphino]-benzene (HMPB) and -ethane (HMPE) exhibited good stability and antitumor activities in vitro and in vivo for cancer research applications [Pillarsetty, N., et al., J. Med. Chem. 2003. 46(7): p. 1130-1132 (2003)]. It suggests that those ligands containing hydroxymethylphosphines can be potentially used in vivo without any severe toxicity or inflammation for biomedical purposes although that cytotoxicity has not been fully elucidated yet. In typical conjugation experiments, reaction mixtures of hydroxymethylphosphines and amines of proteins can be stirred in water or ethanol at room temperature (25° C.) for 1-3 hour to be crosslinked. To the best of our knowledge, there has been no study to determine efficiency and reaction kinetics of conjugation or crosslinking with biological molecules, specifically peptide-based materials, pH effect on this reaction and its detailed mechanism in terms of organic chemistry.

Although a detailed organic chemistry of Mannich type addition of —NH₂ groups to —CH₂OH groups of hydroxymethylphosphines has not been fully elucidated yet, Katti group reported that a reaction of hydroxymethylphosphines of THP with glycines produces the linear structure of aminomethylphosphines with the secondary amines while the mixture of THP with HMPB or HMPE forms two heterocyclic seven-membered ring structures with the tertiary amines due to two hydroxymethylphosphosphines separated by two carbon atoms [Katti, K. V., et al, U.S. Pat. No. 5,948,386].

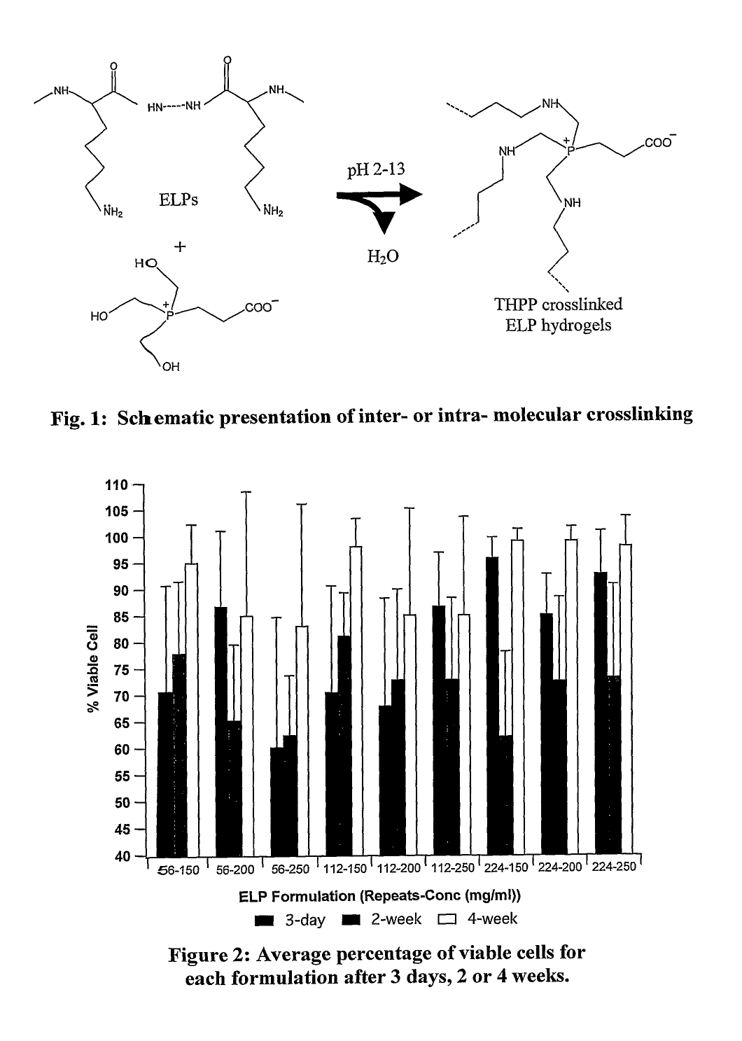

As shown in FIG. 1, we propose here that primary amines are attached to methylene groups (—CH₂) of hydroxymethylphosphines, releasing hydroxyl groups in the form of water, which results in the secondary amines of aminomethylphosphines, which suggests that two or three amines are attached to one THPP molecule in the form of the linear form with the secondary amines considering a potential steric hindrance for crosslinking. There is little probability that after the first Mannich-type condensation, the secondary amines can be further attached to the unreacted, proximal methylene groups (—CH₂) of hydroxymethylphosphines, producing tertiary amines with a highly strained, four-membered ring structure of aminomethylphosphines.

The present invention is explained in greater detail in the following non-limiting Examples.

### EXAMPLE 1

### Cell Viability

Primary porcine chlondrocytes were isolated from the femoral chondyles of skeletally immature pigs. After isolation and washing, cells were concentrated to 100×10⁶ cells per ml in sterile HEPES-buffered saline.

ELP[KV₆-56] (MW=23.9 kDa), ELP[KV₆-112]] (MW=47.1 kDa), and ELP[KV₆-224] (MW=94.3 kDa) were synthesized using a genetic engineering method, where the monomer of the ELP gene was chemically synthesized and oligomerized using recursive directional ligation (RDL) as described by US Patent Chilkoti. The protein was then expressed in E. coli and purified using the inverse transition cycling (ITC) technique in accordance with known techniques (see, e.g., U.S. Pat. No. 6,699,294 to Urry).

The purified proteins were then sterile filtered through 0.2 μm syringe filters and their concentrations measured on a UV spectrophotometer. Three aliquots were taken from each molecular weight of ELP and the concentrations adjusted to 150, 200, or 250 mg/ml so that there were nine different ELP solutions: 3 molecular weights×3 concentrations. Twelve 50 microliter aliquots of each ELP were prepared in 500 microliter microfuge tubes. Fifty milligrams of the crosslinker, β-[Tris(hydroxymethyl)phosphino]propionic acid (betaine) (THPP), was reconstituted in 200 microliters sterile HEPES-buffered saline.

Five microliters of cell suspension were added to each ELP aliquot and dispersed by stirring and pipetting. The required amount of THPP was then added to one tube, so that there was a 6-fold molar excess of hydroxyl groups on the crosslinker to primary amines on the ELP. The crosslinker was dispersed by stirring and pipetting, and the solution was pipetted into custom molds. This was repeated for the remaining eleven ELP aliquots for a given formulation, one at a time, until all formulations had been crosslinked. The filled molds were then placed in a humidified 37° C. incubator for 10 minutes to allow for complete crosslinking. Once turgid, gels were removed from the molds with a flat spatula and placed in separate wells of a 48-well tissue culture plate. Gels were overlaid with 1 ml fresh culture medium. This was repeated for all nine ELP formulations. Hydrogels were cultured for 3 days, 2 weeks, or 4 weeks (n=3, n=1 cell-free control) before being assessed for viability using the LIVE/DEAD Cell Viability and Cytotoxicity Kit (Molecular Probes, Eugene, Oreg.).

At the time of the viability assay, gels to be tested were washed three times (5 minutes each) in sterile HEPES-buffered saline before being incubated for 30 minutes with the LIVE/DEAD reagents. After incubation with assay reagents, gels were once again washed three times (5 minutes per wash) in sterile HEPES-buffered saline. Gels were then imaged on a laser scanning confocal microscope (Zeiss LSM 510, Carl Zeiss, Inc., Thornwood, N.Y.). One gel was treated with 70% ethanol for 10 minutes before being treated with assay reagents to serve as a negative control. Each gel was imaged in five randomly selected areas, and images were processed using a custom-written Matlab code to count live (green) and dead (red) cells within each image. Results are presented as the average percentage of viable cells counted from all images (n=15 per formulation) per gel formulation.

### EXAMPLE 2

### Mechanical Testing of ELP₅'S

ELP hydrogels were crosslinked with THPP. ELP[KV_{—}6-56] (MW=23.9 kDa), ELP[KV_{—}6-112] (MW=47.1 kDa), and ELP[KV_{—}6-224] (MW=94.3 kDa) were aliquoted according to molecular weight and the concentrations adjusted to 150, 200, or 250 mg/ml so that there were nine different ELP solutions: 3 molecular weights×3 concentrations. Five 200 microliter aliquots of each formulation were prepared in 1.5 ml microfuge tubes. Fifty milligrams of the crosslinker, b-[Tris(hydroxylmethyl)phosphino] propionic acid (betaine) (THPP), was reconstituted in 200 microliters sterile HEPES-buffered saline. The required amount of THPP was then added to one ELP aliquot, so that there was a 6-fold molar excess of hydroxyl groups on the crosslinker to primary amines on the ELP. The crosslinker was dispersed by stirring and pipetting, and the solution was pipetted into custom molds. This was repeated for the remaining four ELP aliquots for a given formulation, one at a time, until all formulations had been crosslinked. The filled molds were then placed in a humidified 37° C. incubator for 10 minutes to allow for complete crosslinking. Once turgid, gels were removed from the molds with a flat spatula and placed in separate wells of a 24-well tissue culture plate. “Hydrogels (n=5 per formulation) were tested to determine equilibrium compressive modulus (E), frequency-dependent dynamic shear modulus (G*), loss angle (δ), and equilibrium shear modulus (μ). Mechanical tests were performed on a strain-controlled rheometer (ARES; Rheometrics, Piscataway, N.J.) in a parallel plate configuration using stainless steel platens, with the sample and test platens submerged in a temperature-controlled, circulating water bath held at 37° C. Samples were equilibrated with a tare load of 5-7 grams and allowed to relax to equilibrium. The reference thickness was recorded as the distance between platens under the compressive tare load. Samples were compressed to 5% compressive strain (c) and allowed to relax for to equilibrium. This protocol was repeated in 5% increments until 20% compression was achieved. Linear regression of calculated normal stress (τ) at equilibrium on ε was performed to calculate an equilibrium compressive modulus (E_{eq}). At 20% compression, a dynamic frequency sweep was performed in torsional shear at a maximum shear strain of 0.05 (0.1-50 rad/sec). Linear viscoelastic theory was used to calculate the magnitude of the complex shear modulus (|G*|) and loss angle (δ), representing stiffness and internal dissipation of the material under dynamic loading, respectively. Samples were then tested in torsional shear at γ=0.05 strain increments from γ=0.05-0.20 to determine an equilibrium shear modulus (μ). Samples were allowed to relax for 10 minutes at each step, at which time, they had reached equilibrium. All experiments were performed in distilled water.

### EXAMPLE 3

### Oscillatory Rheological Test for Crosslinking

Two new elastin-like polypeptides (ELPs) were designed from Val-Pro-Gly-Xaa-Gly (Xaa: is a guest residue and may be any amino acid other than Pro): ELP[KV7F-72, 144] and ELP[KV2F-64, 128], where the frequency of the guest residue and the number of the repeated pentapeptides are expressed in a bracket. ELPs were genetically synthesized, polymerized in E. coli, and purified by inverse transition cycling (ITC) in accordance with known techniques (see, e.g., U.S. Pat. No. 6,699,294 to Urry). Purified ELPs were dialyzed against water at 4° C. for 3 days to completely remove salt and phosphates, and then freeze-dried for the crosslinking with THPP,β-[tris(hydroxylmethyl) phosphino]-propionic acid in phosphate buffer at pH 7.5 and 12.0. Oscillatory Theological tests were performed when 20 (wt-)% THPP crosslinker in phosphate buffer (pH7.5) at 700 mM of NaCl was added to 20 (wt-)% of ELP [KV7F-144] in phosphate buffer (pH 7.5) prepared onto a bottom platen of which temperature was equilibrated at 35° C. The Theological behaviors during crosslinking were characterized in dynamic, torsional shearing using a cone-on-plate configuration (ARES Rheometer, TA Instruments, cone angle=0.1 rad, plate radius=25 mm) at 35° C. for 1 hr (1 Hz frequency and 0.01 maximum shear strain).

### EXAMPLE 4

### Crosslinking Solvent pH Effects on ELP Mechanics

Proposed novel applications of the crosslinking strategy include the manipulation of solvent viscosity, temperature, pH and/or ionic strength to kinetically control the crosslinking reaction, and also the introduction of new kinds of ELPs with different frequencies of crosslinkable Lys guest residue and modified crosslinking pH in aqueous solution to modulate crosslinking densities and mechanical properties. In this example, ELP hydrogels were crosslinked with THPP in phosphate buffers of two different pH values (pH 7.5 or 12.0) in order to determine the effect of this parameter on crosslinked polymer mechanical properties. Two new elastin-like polypeptides (ELPs) with different crosslinkable Lys residues were designed; ELP[KV₇F-72] (MW=31.0 kDa) and ELP[KV₂F-64] (MW=28.3 kDa), where the frequency of the guest residue and the number of the repeated pentapeptides are denoted in the bracket. As described before, ELPs were genetically synthesized, expressed in E. coli and thermally purified. The purified ELPs were dialyzed against water at 4° C. for 3 days to remove salts and free phosphates, which were then freeze-dried and reconstituted in 20 mM phosphate buffers (pH of 7.5 or 12) to give a protein concentration of 200 mg/ml. The crosslinker (THPP) was then added to 150 μl ELP solution to yield a molar excess of 8 to 9 amine reactive sites and 100 mM NaCl. The mixture was placed in a customized Teflon mold (8 mm of diameter and 2 mm of depth) and incubated at 37° C. for 1 hr.

The resulting hydrogels (four groups: 2 pH×2 ELP formulations; n=4 per group) of ELP [KV7F-72] and ELP[KV2F-64] were then tested to determine equilibrium compressive modulus (E), complex shear modulus (G*), loss angle (δ), and equilibrium shear modulus (μ) as described in Example 2. Mechanical tests were performed on a strain-controlled rheometer (ARES; Rheometrics, Piscataway, N.J.) in a parallel plate configuration. Porous and stainless steel parallel plate platen were used (plate radius=10 mm; stainless steel porous platens; 50% porosity; 40-60 micron pore size), with the sample and test platens submerged in a temperature-controlled, circulating water bath held at room temperature (˜23° C.) Samples were equilibrated with a tare load of 5-10 grams and allowed to relax until equilibrium (1 to 3 hours). The reference thickness was recorded as the distance between platens under the compressive tare load. Samples were compressed to 5% compressive strain (ε) and allowed to relax until equilibrium. This protocol was repeated in 5% increments until 15% was achieved. Linear regression of calculated normal stress (τ) at equilibrium on ε was performed to calculate an equilibrium compressive modulus (E_{eq}). At 15% compression, a dynamic frequency sweep was performed in torsional shear at a maximum shear strain of 0.01 (0.1-50 rad/sec). Linear -viscoelastic theory was used to calculate the magnitude of the complex shear modulus (|G*|) and loss angle (δ), representing stiffness and internal dissipation of the material under dynamic loading, respectively. Samples were then tested in torsional shear at γ₀=0.05 strain increments from 0.05 to 0.15 to determine equilibrium shear modulus (μ). Samples were allowed to relax for 5 minutes at each step. All experiments were performed in phosphate buffer at pH 7.5.

### EXAMPLE 5

### Results: Cell Viability

FIG. 2 shows results for the 4-week cell viability tests conducted in nine different formulations of ELP[KV₆]. Cell viability showed average values between 70 and 90% for all formulations at all time points. There was no evidence of an effect of molecular weight or concentration on cell viability at any time point.

### EXAMPLE 6

### Results: Mechanical Testing of ELP₅'S

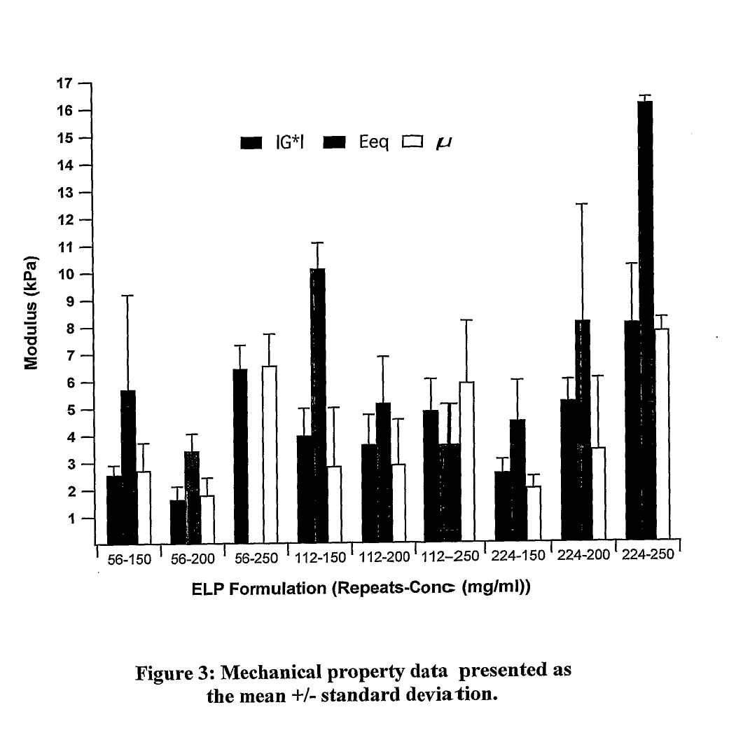

FIG. 3 shows results for mechanical testing of the ELP[KV₆] hydrogels. The equilibrium shear modulus and magnitude of complex shear modulus increased with increasing concentration for each molecular weight formulation. There was also a general trend toward increasing values with increased molecular weight. Values for either μ and |G*| fall within ranges reported previously for nucleus pulposus, articular cartilage and meniscus (Table 1). Further increases in shear moduli may be expected by increasing the number of lysine residues on the ELP molecule, and we have also observed that the pH of the crosslinking solvent influences the mechanical properties of the resulting hydrogels (Example 3).

### EXAMPLE 7

### Results: Oscillatory Rheological Testing of ELPs

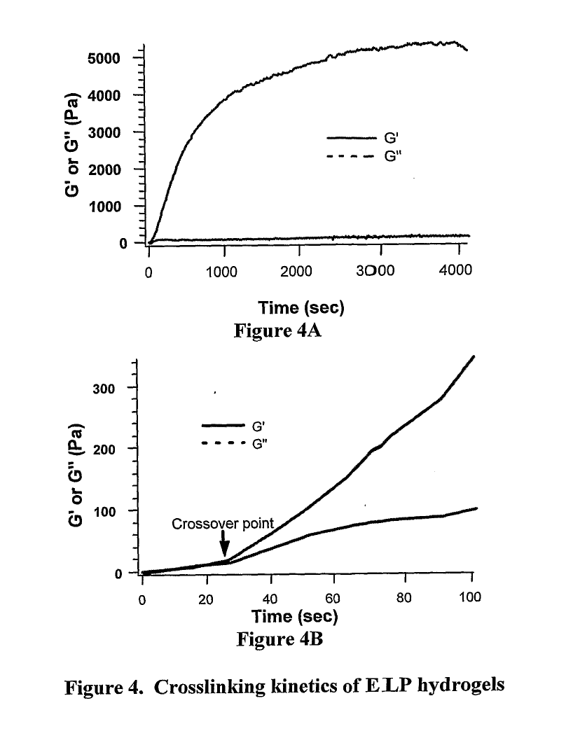

As shown in FIGS. 4A-4B for ELP[KV7F-144], the crosslinking of ELPs at 35° C. came to completion within one minute through the condensation of Lys residues and THPP. The crossover point, where the elastic response of the hydrogel starts to dominate the viscous response, was observed within 30 sec of the reaction time as indicated by the crossover of G′ and G″. Values for the storage modulus, G′, were observed to increase to more than 5 kPa within one hour. These results demonstrate that ELP hydrogels crosslinked with THPP formed rapidly in aqueous solution, and that crosslinking densities and mechanical properties can be largely controlled by both Lys residues of ELPs and crosslinking pH. These observations are useful for the design of a diverse array of injectable and in situ polymerizing biomaterials.

### EXAMPLE 8

### Results: pH Effects on ELP Mechanics

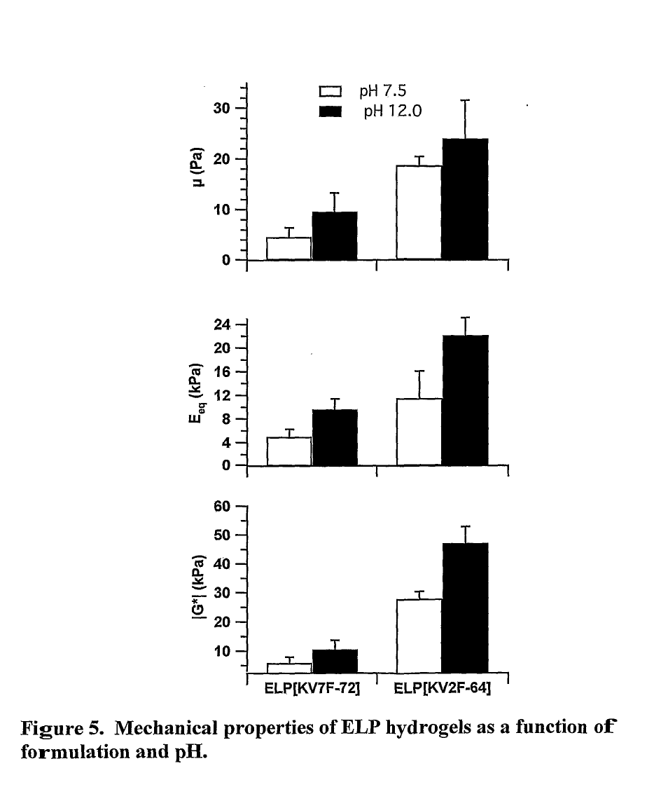

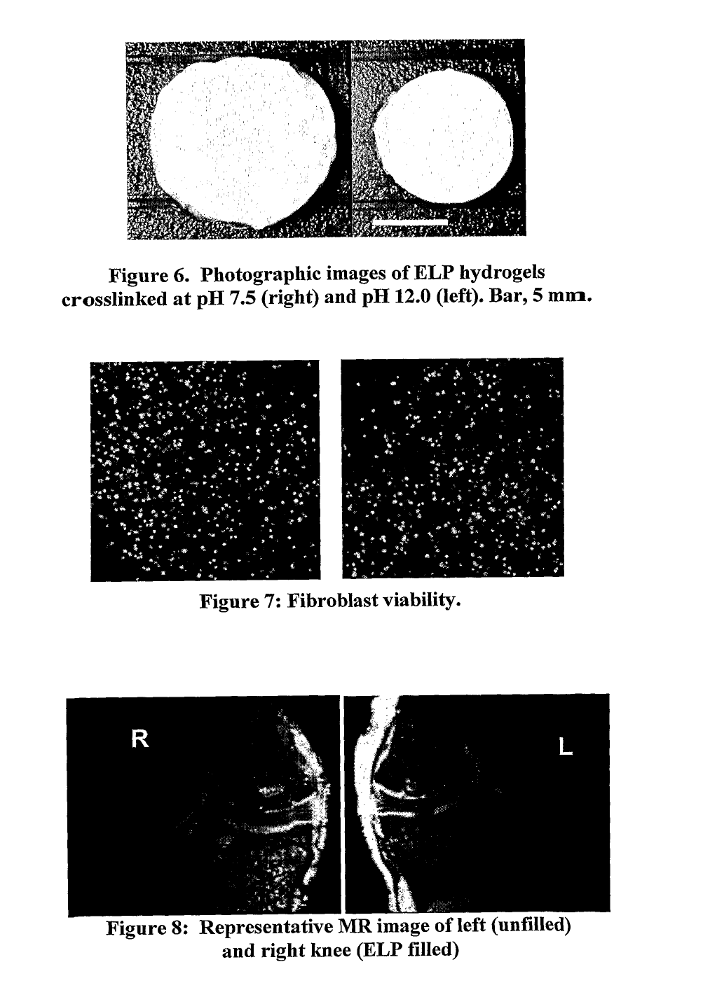

Values for |G*|, E_{eq} and μ were found to differ between formulations and the crosslinking solvent pH conditions. As shown in FIGS. 5A-5C, average values for |G*| varied from 5.8 to 46.9 kPa between the formulations and the crosslinking solvent pH, and were higher at pH 12.0 as compared to pH 7.5 with the same ELP formulations, which may result from different ratios of protonation/deprotonation of primary amines of Lys residue (pKₐ(ε-NH₂ of Lys)=10.54) based on the crosslinking pH. In addition, values for |G*| of the ELP[KV2F-64] gel were higher than that of the ELP[KV7F-72] gel under all pH conditions because ELP[KV2F-64] has higher frequency of crosslinkable amine group of Lys guest residue than ELP[KV7F]. This general pattern was observed for E_{eq} and μ as well, with higher stiffnesses reported for the higher pH crosslinking solvent. Visual examination of the crosslinked hydrogels also revealed substantial differences in their crosslinking properties (FIG. 6). It is suggested that both the frequency of Lys guest residue of ELPs and the ratio of protonation/deprotonation of its amines based on crosslinking pH largely determine crosslinking properties and mechanical properties. The highest value for equilibrium compressive and shear modulus of all studied ELPs and crosslinking solvents was for the pH 12.0 crosslinking solvent, and well within the range of physical properties reported for intervertebral disc and meniscus.

### EXAMPLE 9

### Diffusion Assays in Crosslinked ELPs

Crosslinked hydrogels of ELP[KV_{—}6-112] were prepared from ELP₅ crosslinked with THPP as described in Example 2. One hydrogel sample was divided into four sections and incubated in solutions of 70 kDa molecular weight, uncharged fluorescein-conjugated dextrans (Molecular Probes, Eugene, Oreg.). The dextran was suspended in DMEM/Nutrient Mixture F-12 Ham, phenol red-free media at 2.27 mg/mL (0.032 mM). Gel sections were incubated in the dextran solution for 96 hours at 37° C. to allow full permeation of the sample. Fluorescence recovery after photobleaching (FRAP) experiments were performed to quantify the molecular diffusion coefficients (Leddy and Guilak, 2003). Briefly, samples were placed in a custom-built chamber that held each hydrogel against a cover slip atop the stage of a laser scanning confocal microscope (Zeiss LSM 510, Carl Zeiss, Inc., Thornwood, N.Y.). The chamber was filled with the phenol red-free media. Photobleaching of the imaged area was performed (488 nm emission at 100% laser power) followed by imaging to determine the radius of fluorescence recovery and the half-recovery time (20×/0.5 NA Plan-Neofluar, optical slice <7.2 μm, excitation/emission 488/505 nm). Image processing was performed to calculate the diffusion coefficient (D) according to the method of Axelrod and co-workers (1976), using the mean radius of the bleached region (ω), the half recovery time (τ_{1/2}), and a bleaching parameter (γ_{D}) according to the equation, D=(ω²/4τ_{1/2})γ_{D}. Five FRAP experiments were performed on each sample.

### EXAMPLE 10

### Cell Viability—Fibroblasts in Vitro

Crosslinked ELP block copolymer hydrogels were examined using fibroblasts to see their ability to support cell viability. ELP block copolymers were mixed with mouse fibroblasts (NIH-3T3, 10⁷ cells/ml) and THPP at a 1× molar excess of —OH to —NH₂. Cell viability was assessed at day 0 and 3 after culture using Live/Dead and PicoGreen assay kits (Molecular Probes). When fibroblasts were mixed with various ELP block copolymers and THPP during crosslinking, ELP hydrogels were formed after incubation at 37° C. within several minutes. FIG. 7 shows that most fibroblasts were stained green in the cell viability assay, suggesting the crosslinking reaction was not cytotoxic to fibroblasts at 3 day, but cell viability based on DNA quantitation was found to be less than 50% immediately after crosslinking, but was greater than 60% after 3 days of culture, values that compare well with embedding cells in alginate or agarose. These results may arise as the THPP is capable of reacting with cell-associated amines; however, shortly after encapsulation, the cells appear to recover or proliferate in a process that does not interfere with long-term cell survivability.

### EXAMPLE 11

### Osteochondral Defect-Filling in a Goat Model of Cartilage Repair

ELP[KV₆-224] (MW=94.3 kDa) was expressed and purified as described in Example xx above. The purified proteins were then sterile filtered through 0.2 μm syringe filters and their concentrations measured on a UV spectrophotometer. The concentration was adjusted to 200 mg/ml and store in sterile container. Fifty milligrams of the crosslinker, β-[Tris(hydroxymethyl)phosphino] propionic acid (betaine) (THPP), was reconstituted in 200 microliters sterile HEPES-buffered saline.

Adult female Nubian goats (>90 kg) underwent bilateral knee surgery for creation of a cylindrical osteochondral defect (6 mm dia.×4 mm depth) in the medial condyle. All surgery was performed in a certified veterinary operating suite at Duke University Medical Center under an IACUC-approved protocol. A 10.14 microliter volume of THPP was added to a sterile 300 microliter volume of ELP in the operating suite and dispersed by stirring. The mixed solution was drawn into an 18 gauge syringe in preparation for filling the critically-sized defects. Approximately 150 microliters was delivered to defects in order to create a proud fill. The mixture was observed to form a turgid gel within 5 minutes of injection. Seven goats received osteochondral defects on both medial femoral condyles. One defect in each animal was filled with the ELP solution, while the other defect was left empty and served as an internal control. Three goats were sacrificed at 3 months, and 4 goats were sacrificed at 6 months. Upon sacrifice, joints were imaged using high-resolution MRI and images were scored according to a semiquantitative scale (Table 4). Joints were then opened and scored for gross appearance before being processed for routine histology and semi-quantitative histological scoring (Table 4).

TABLE 4
Semi-quantitative grading schemes used to visually assess
osteochondral repair via MRI and histological staining

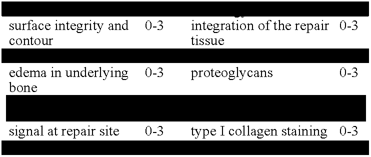

### EXAMPLE 12

### Osteochondral Defect-Filling in a Goat Model of Cartilage Repair

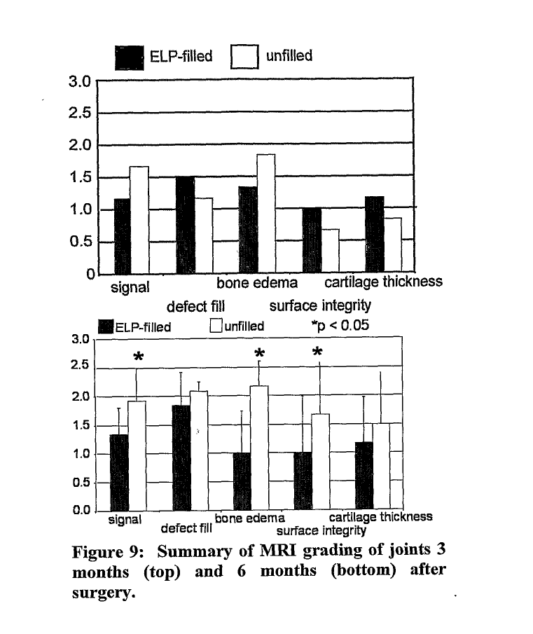

All animals were returned to cage activity followed by free range activity upon a farm immediately after surgery. No complications associated with the right or left knee joints of any animals occurred following surgery to the 6 months timepoint. At 3 and 6 mos. after surgery, the site of the defects was evident as increased signal intensity upon MR imaging, particularly in the underlying bony areas (FIG. 8). Some changes to the cartilage were also evident, as suggested by MRI grading (FIG. 9). Upon MRI, joints filled with ELP had significantly lower scores for (significantly less evidence of) MR signal, edema, and surface integrity as compared to control defects. It was not possible to identify any residual polymer via differences in MRI parameters at either 3 or 6 months after surgery using the GRE sequence chosen here. There was no evidence of osteophyte formation or inflammation in the joints at either timepoint.

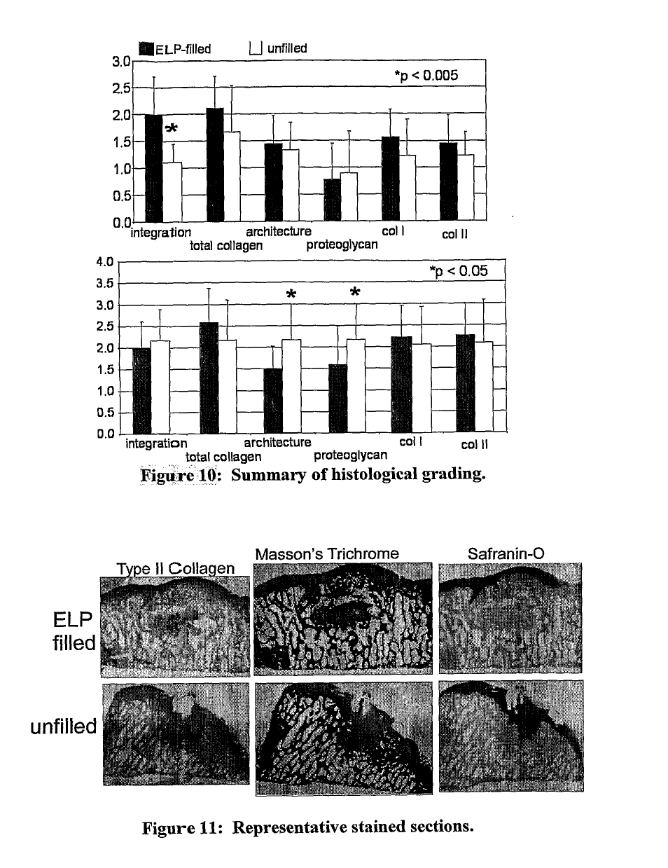

At 3 months after surgery, histological grading revealed significantly higher scores for integration of ELP-filled defects, as well as greater evidence of total collagen staining, lesser type I and more type II collagen staining (FIG. 10, top). By 6 months after surgery, histological grading revealed that ELP-filled defects scored higher for total collagen, but lower in ELP-filled defects for architecture and proteoglycan (FIG. 10, bottom). Histological examination at both timepoints revealed that cell infiltration was not apparently inhibited in the ELP-filled defects, nor was there any evidence of inflammation. It was not possible to definitively conclude that ELP was present or absent at the 3 or 6 month timepoint upon histological assessment; in vitro degradation data suggested that the crosslinked ELP scaffolds may be degradable in vivo.

FIG. 11 shows representative stained sections of cartilage from one goat at 6 months after surgery. Defect sites are evident where underlying bone is more disorganized and/or of increased density.

These results suggest that more rapid defect healing occurred at the 3 month timepoint in the ELP filled osteochondral defects, as evidenced by improved integration and more cartilage-like tissue appearance. Some of these characteristics were not retained at the 6 month timepoint, and differences in bony architecture and proteoglycan staining became evident between the ELP-filled and unfilled defects. While MRI is the preferred clinical assessment tool, it is noteworthy that MRI appearance was not coordinated with histological appearance in a consistent manner.

The foregoing is illustrative of the present invention, and is not to be construed as limiting thereof. The invention is defined by the following claims, with equivalents of the claims to be included therein.

That which is claimed is:

1. A method of filling a defect in a cartilaginous tissue, comprising the steps of:
(a) mixing:
(i) a first reagent composition comprising an amine-free hydroxymethyl phosphine crosslinking agent with
(ii) a second reagent composition comprising a bioelastic polymer, said bioelastic polymer comprising elastomeric units selected from the group consisting of bioelastic pentapeptides, tetrapeptides, and nonapeptides;
to produce a therapeutic composition; and then
(b) administering said therapeutic composition to said cargilagenous tissue in an amount sufficient to at least partially fill said defect with said therapeutic composition, with said elastin-like polypeptides crosslinking within said defect.

2. The method of claim 1, wherein said elastomeric unit is a pentapeptide.

3. The method of claim 1, wherein said elastomeric unit is a tetrapeptide.

4. The method of claim 1, wherein said elastomeric unit is a nonapeptide.

5. The method of claim 1, wherein said elastomeric unit comprises a VPGXG repeating unit, where X is any amino acid.

6. The method of claim 1, wherein said cross-linking agent has a free carboxylic acid group.

7. The method of claim 1, wherein said cross-linking agent has a compound of interest coupled thereto.

8. The method of claim 1, wherein said crosslinking agent is selected from the group consisting of B-(tris-(hydroxymethyl)phosphino)propionic acid (THPP) and tris(hydroxymethyl)phosphine (THP).

9. The method of claim 1, wherein said bioelastic polymer when crosslinked is characterized by:
(i) a complex modulus |G*| of 0.1 to 700 kPa;
(ii) a loss angle δ of 1 to 50°; and
(iii) an equilibrium shear modulus μ of 0.1 to 500 kPa.

10. The method of claim 1, wherein said tissue is an articular cartilage.

11. The method of claim 10, wherein said bioelastic polymer when crosslinked is characterized by:
(i) a complex modulus |G*| of 2-400 kPa;
(ii) a loss angle δ of 2-20°; and
(iii) an equilibrium shear modulus μ of 2-400 kPa.

12. The method of claim 11, wherein said bioelastic polymer when crosslinked is further characterized by:
(iv) a compressive modulus in unconfined compression E of 5-2000 kPa; and
(v) a compressive modulus in uniaxial compression H_{A} of 5-1000 kPa.

13. The method of claim 12, wherein said bioelastic polymer when crosslinked is further characterized by:
(vi) a diffusion coefficient for 70 kDa solutes of greater than 25 μm²/s.

14. The method of claim 1, wherein said tissue is a meniscus.

15. The method of claim 14, wherein said bioelastic polymer when crosslinked is characterized by:
(i) a complex modulus |G*| of 2-650 kPa;
(ii) a loss angle δ of 2-30°; and
(iii) an equilibrium shear modulus μ of 2 to 500 kPa.

16. The method of claim 15, wherein said bioelastic polymer when crosslinked is further characterized by:
(iv) a compressive modulus in unconfined compression E of 5-100 kPa; and
(v) a compressive modulus in uniaxial compression H_{A} of 5-500 kPa.

17. The method of claim 1, wherein said tissue is an intervertebral disc.

18. The method of claim 17, wherein said tissue comprises a nucleus pulposus.

19. The method of claim 18, wherein said bioelastic polymer when crosslinked is characterized by:
(i) a complex modulus |G*| of 0.1-70 kPa;
(ii) a loss angle δ of 2-50°; and
(iii) an equilibrium shear modulus μ of 0.1 to 30 kPa.

20. The method of claim 19, wherein said bioelastic polymer when crosslinked is further characterized by:
(iv) a diffusion coefficient for 70 kDa solutes of greater than 5 μm²/s.

21. The method of claim 17, wherein said tissue comprises an anulus fibrosus.

22. The method of claim 21, wherein said bioelastic polymer when crosslinked is characterized by:
(i) a complex modulus |G*| of 1-400 kPa;
(ii) a loss angle δ of 2-40°; and
(iii) an equilibrium shear modulus μ of 2-210 kPa.

23. The method of claim 22, wherein said bioelastic polymer when crosslinked is further characterized by:
(iv) a compressive modulus in uniaxial compression H_{A} of 5-1000 kPa.

24. The method of claim 23, wherein said bioelastic polymer when crosslinked is further characterized by:
(v) a diffusion coefficient for 70 kDa solutes of greater than 5 μm²/s.

25. The method of claim 1, wherein said therapeutic composition comprises a hydrogel.

26. The method of claim 1, wherein said filling step is carried out within two hours of said mixing step.

27. A kit useful for making a composition for filling defects in cartilageneous tissue, comprising:
(i) a first reagent composition comprising an amine-free hydroxymethyl phosphine crosslinking agent; and
(ii) a second reagent composition comprising a bioelastic polymer, said bioelastic polymer comprising elastomeric units selected from the group consisting of bioelastic pentapeptides, tetrapeptides, and nonapeptides.

28. The kit of claim 27, wherein said bioelastic polymer when crosslinked is characterized by:
(i) a complex modulus |G*| of 0.1 to 700 kPa;
(ii) a loss angle δ of 1 to 50°; and
(iii) an equilibrium shear modulus μ of 0.1 to 500 kPa.

29. The kit of claim 27, wherein said tissue is an articular cartilage.

30. The kit of claim 29, wherein said bioelastic polymer when crosslinked with said crosslinking agent is characterized by:
(i) a complex modulus |G*| of 2-400 kPa;
(ii) a loss angle δ of 2-20°; and
(iii) an equilibrium shear modulus μ of 2-400 kPa.

31. The kit of claim 30, wherein said bioelastic polymer when crosslinked with said crosslinking agent is further characterized by:
(iv) a compressive modulus in unconfined compression E of 5-2000 kPa; and
(v) a compressive modulus in uniaxial compression H_{A} of 5-1000 kPa.

32. The kit of claim 31, wherein said bioelastic polymer when crosslinked with said crosslinking agent is further characterized by:
(vi) a diffusion coefficient for 70 kDa solutes of greater than 25 μm²/s.

33. The kit of claim 27, wherein said tissue is a meniscus.

34. The kit of claim 33, wherein said bioelastic polymer when crosslinked with said crosslinking agent is characterized by:
(i) a complex modulus |G*| of 2-650 kPa;
(ii) a loss angle δ of 2-30°; and
(iii) an equilibrium shear modulus μ of 2 to 500 kPa.

35. The kit of claim 34, wherein said bioelastic polymer when crosslinked with said crosslinking agent is further characterized by:
(iv) a compressive modulus in unconfined compression E of 5-100 kPa; and
(v) a compressive modulus in uniaxial compression H_{A} of 5-500 kPa.

36. The kit of claim 27, wherein said tissue is an intervertebral disc.

37. The kit of claim 36, wherein said tissue comprises a nucleus pulposus.

38. The kit of claim 37, wherein said bioelastic polymer when crosslinked with said crosslinking agent is characterized by:
(i) a complex modulus |G*| of 0.1-70 kPa;
(ii) a loss angle δ of 2-50°; and
(iii) an equilibrium shear modulus μ of 0.1 to 30 kPa.

39. The kit of claim 38, wherein said bioelastic polymer when crosslinked with said crosslinking agent is further characterized by:
(iv) a diffusion coefficient for 70 kDa solutes of greater than 5 μm²/s.

40. The kit of claim 36, wherein said tissue comprises an anulus fibrosus.

41. The kit of claim 40, wherein said bioelastic polymer when crosslinked with said crosslinking agent is characterized by:
(i) a complex modulus |G*| of 1-400 kPa;
(ii) a loss angle δ of 2-40°; and
(iii) an equilibrium shear modulus μ of 2-210 kPa.

42. The kit of claim 41, wherein said bioelastic polymer when crosslinked with said crosslinking agent is further characterized by:
(iv) a compressive modulus in uniaxial compression H_{A} of 5-1000 kPa.

43. The kit of claim 42, wherein said bioelastic polymer when crosslinked with said crosslinking agent is further characterized by:
(v) a diffusion coefficient for 70 kDa solutes of greater than 5 μm²/s.

44. The kit of claim 27, wherein said cross-linking agent has a free carboxylic acid group.

45. The kit of claim 27, wherein said cross-linking agent has a compound of interest coupled thereto.

46. A sterile therapeutic composition produced by the process of mixing (i) a first reagent composition comprising an amine-free hydroxymethyl phosphine crosslinking agent with (ii) a second reagent composition comprising a bioelastic polymer, said bioelastic polymer comprising elastomeric units selected from the group consisting of bioelastic pentapeptides, tetrapeptides, and nonapeptides, to produce said therapeutic composition.

47. The composition of claim 46, wherein said cross-linking agent has a free carboxylic acid group.

48. The composition of claim 46, wherein said cross-linking agent has a compound of interest coupled thereto.
